(19) United States
(12) Patent Application Publication
(10) Pub. No.: US 2005/0196812 A1
(43) Pub. Date: Sep. 8, 2005

(54) Determining the estimated date of embryo implantation and related dates

(76) Inventor: David Edward Williams, Girton (GB)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/074,059

(22) Filed: Mar. 7, 2005

Related U.S. Application Data
(60) Provisional application No. 60/563,592, filed on Apr. 20, 2004.

(30) Foreign Application Priority Data
Mar. 5, 2004 (EP) 04251289.7

(51) Int. Cl. G01N 33/53; G06F 19/00; G01N 33/48

(52) U.S. Cl. 435/7.1; 702/19

(57) ABSTRACT

A method of determining the estimated date of implantation of an embryo may include testing a sample of a body fluid from a pregnant human female subject so as to obtain data indicative of the concentration of human chorionic gonadotrophin (hCG) in the sample, and calculating an estimated date of implantation from the hCG concentration data.

### CROSS-REFERENCE TO RELATED APPLICATION

This application claims the benefit of U.S. Provisional Application No. 60/563,592, filed Apr. 20, 2004, and also claims right of priority to European Patent Application Ser. No. EP04251289.7, filed Mar. 5, 2004.

### FIELD

The present disclosure relates to determining the implantation date of an embryo for the birth of a mammalian subject. The disclosure also relates to determining the estimated due date (EDD) for the birth and the probable date of conception.

### INTRODUCTION

The estimated due date (EDD) is the date around which birth can normally be expected. In practice, for humans, as little as 5% of births occur actually on the EDD, but around 90% occur within the interval of ten days on either side of the EDD. The EDD is very important as many factors, both clinical and non-clinical, depend on it (e.g. arrangement of appointments with obstetricians, decisions to induce labor etc).

At present, two methods of determining the EDD are conventionally employed. The simplest is the “LMP” (or “last menstrual period”) method. This requires knowledge of the date of the woman's last period. In the LMP method the EDD may be determined by taking the first day of bleeding in the last period and either (a) adding 7 days and subtracting 3 calendar months (and advancing the year) or (b) adding 280 days (10 lunar months of exactly 28 days). The result of either calculation is essentially similar although the precise result may vary by a day or two because calendar months have different numbers of days.

The LMP method of EDD determination has the advantage of technological simplicity. However, it may not always be reliable (e.g. where a woman cannot remember accurately the date of her most recent period, or experiences highly irregular cycles).

As an alternative, in circumstances where the LMP method may not be appropriate, or as confirmation of an EDD determined by the LMP method, an ultrasound scan may be performed. The size of the fetus can be used to determine the EDD. However, the accuracy of EDDs determined by ultrasound examination is significantly reduced if the scan is performed after the 13^{th} week of pregnancy. Moreover, EDD determination by ultrasound examination requires a skilled practitioner and the use of expensive and cumbersome equipment. It would be advantageous to provide an accurate method of EDD determination which can be performed simply.

It has not, to the knowledge of the inventor, hitherto been suggested that the EDD could be determined on the basis of hormonal measurements.

### SUMMARY

In an embodiment, a method of determining the estimated date of implantation of an embryo may include testing a sample of a body fluid from a pregnant human female subject so as to obtain data indicative of the concentration of human chorionic gonadotrophin (hCG) in the sample, and calculating an estimated date of implantation from the hCG concentration data.

In an embodiment, a device for determining the estimated implantation date of an embryo may include a data processor responsive to hCG concentration data to calculate the estimated implantation date.

The sample of body fluid could be any body fluid in which representative levels of hCG are present (e.g. blood, plasma, serum). A preferred body fluid is urine. Methods of determining the concentration of hCG in urine are well known to those skilled in the art.

### BRIEF DESCRIPTION OF THE FIGURES

The subject matter will now be further described, along with illustrative examples, and with reference to the accompanying drawings, in which:

FIGS. 1 and 2 are graphs showing concentration of hCG in urine (in mIU/ml) against time (days post implantation); and

FIG. 3 shows a perspective view of an electronic assay result reading device suitable for use in performing the disclosed methods.

### DETAILED DESCRIPTION

The disclosed methods may involve determination of the concentration of hCG in a sample of body fluid. The hCG concentration may be determined in a relative manner (e.g. by comparison with a reference or threshold value) but it is much more preferred that the concentration of hCG be determined in an absolute manner (e.g. by measuring the number of mIU of hCG per ml of fluid sample).

Known methods of assaying hCG in samples of body fluid include immunological techniques (i.e. the use of an immunoglobulin, especially a monoclonal antibody, having specific binding for hCG), and such techniques may advantageously be employed with the devices and methods disclosed herein. In particular it is preferred to use an assay technique which can be performed by the woman herself at home, or performed simply by a general practitioner, without resort to laboratory equipment or analysis. In this regard lateral flow assay devices are especially useful, being cheap and simple to use.

Many lateral flow assay devices are known which are suitable for measurement of hCG in urine, as these constitute the basis of most home pregnancy test kits. Examples of such devices are disclosed in EP 0291194.

In general, however, these devices are not ideally suited for use in the presently disclosed methods, as they provide only a qualitative assay result (i.e. “pregnant” or “not pregnant”), and a quantitative result may be preferred. Although it has been known for decades that hCG concentrations rise steeply early in pregnancy (and therefore act as a reliable diagnostic indicator of pregnancy) it has never been proposed that hCG concentration could be used to calculate an estimated date of conception and/or an estimated due date.

Typically home pregnancy test kits involve the use of a dipstick-type lateral flow device impregnated with a labeled hCG-specific immunological reagent which is caused, in the presence of an hCG-containing sample, to accumulate in a test result zone. The label is typically a direct label, such as a colored bead, or colloidal gold. Visual inspection of the device by the user is necessary, and the user must interpret the assay result based on the amount of label accumulated in the test zone.

One type of hCG assay device is provided with a corresponding reader (the CLEARBLUE EASY® Digital Pregnancy Test device, available from Unipath Limited, Bedford, UK) which includes optical measurement systems which can detect and measure the amount of label accumulated. The assay reading device may include an LCD window in which to display the assay result. Such an assay device/assay result reader combination could, suitably programmed, be used in the disclosed methods.

The disclosed methods are such that the EDD and/or estimated date of conception can be calculated from a measurement of hCG concentration obtained from a single sample of body fluid obtained on a single day. If desired, however, hCG concentration may be determined in samples obtained from the subject on two or more days. Determining the hCG concentration in samples obtained on two or three days can, to some extent, enhance the accuracy of the prediction, while determining hCG concentration in samples obtained on four days can provide a more marked improvement in accuracy. In embodiments in which samples of body fluid are obtained on a plurality of days, the sample may be taken at about the same time of day (e.g. +/−1 hour) on each occasion, to reduce variation in sample composition. Where the body fluid sampled is urine, for instance, it is advantageous to perform the hCG assay each day on early morning urine samples, so as to reduce variability due to differences in volume of urine output. If a plurality of samples (obtained on different days) is tested, the samples may be taken on successive days or, in the alternative, a hiatus of one or more days may occur between sampling days, provided that the last day of sampling is within the overall test window.

The hormone concentration is used to estimate the date on which the embryo has been implanted in the uterus. The estimated due date and estimated date of conception may be calculated from the estimated implantation date. The implantation date is estimated by consideration of the hCG levels in conjunction with an empirically determined equation to determine the day on which the hormone concentration started to rise. This day is taken as the estimated day of implantation.

Especially surprising is the finding that one hCG concentration data point can be sufficient to determine date of implantation with reasonable accuracy, especially if the sample is taken early during pregnancy (up to about 15 days post-implantation).

Two sample points can improve accuracy (e.g. in reducing calibration errors in the assay system), especially if the sample points are separated by a hiatus of at least 48 hours. Advantageously, if just two sample points are employed, samples are taken at least 5 days apart, conveniently about 6 or 7 days apart (e.g. one sample at about 15 days post-implantation and another sample at about 22 days post-implantation).

Accuracy may be further improved by taking at least three, or at least four, samples. The samples may be taken on successive days but there may be a temporal separation of at least about 48 hours between the taking of at least two of the samples. The samples may be taken at either regular or irregular intervals, but each (or at least the majority) should be taken at about the same time of day. For example, samples might be taken at days 10, 15, 20 and 25 post-implantation, or at days 15, 17, 24 and 26 post-implantation. It should be borne in mind, however, that maximum accuracy in determination of EDD and/or estimated date of conception is not always necessary and for many purposes, calculations based on just one or two data points may be sufficient.

As mentioned above, immunological techniques for determining the concentration of hCG are especially preferred. The properties of immunological reagents are not universal, e.g. different hCG-specific antibodies will have different binding affinities. Accordingly, it is not appropriate to define the disclosed methods by reference to any particular algorithm, but those skilled in the art, with the benefit of the present disclosure, will readily be able to ascertain an appropriate algorithm for a particular assay system of interest depending, for example, on the antibody or other reagent used, the label, the detection/measurement system etc.

There is an exponential rise in the level of hCG early in pregnancy, in both serum and urine samples. A study of the hCG concentration in urine from a large sample of pregnant women showed that while the hCG concentration on a particular day post-ovulation varies markedly from individual to individual (as might be anticipated), somewhat surprisingly, once hCG is present above background, the value of hCG was found to be generally consistent across the sample as a function of time from the first rise above background. Consequently, hCG concentration can be used to extrapolate “backwards” to provide an estimated date for first appearance of hCG above background in the test sample, which corresponds closely to the date of implantation, which could in turn allow determination of an estimated due date and/or an estimated date of conception (conception generally takes place 7 days, +/−2 days, prior to implantation; and the EDD date can be determined as date of implantation plus 37 weeks).

The exponential increase in hCG concentration following implantation continues for about a further four or five weeks, after which time the hCG concentration tends to plateau, so that hCG measurements made outside this time interval cannot be used to determine accurately the estimated date of implantation.

Accordingly, one or (preferably) more tests of hCG concentration can be made within about 8 weeks, or within about 7 weeks, or within about 6 weeks, of the woman's last menstrual period although, unlike the LMP method of EDD determination, there is no requirement for the woman to recall the precise date of her last period.

The disclosed methods may provide an EDD which is more accurate than that provided by the LMP method, as the present methods are based on physiological data. Unlike the ultrasound scan method, the present methods can be performed very easily, do not require the use of expensive equipment, do not require a high degree of skill and training, and can be performed by the pregnant woman herself, another person, or a health professional without the need for an experienced ultrasonographer.

The present methods may be adapted to measure hCG concentration accurately across a wide range of values, because the hCG concentration increases so rapidly over a short period of time, from 0 mIU/ml at the time of implantation to about 1,000 mIU/ml (in urine) by about two weeks after implantation. If necessary, hCG tests conducted shortly after implantation could be performed using one particular measurement technique, while hCG tests conducted later could be performed using a different measurement technique. It is preferred however to employ a single technique which is suitable for hCG measurements made at any time during the relevant test window of up to 6-8 weeks following the last menstrual period. Techniques for measurement of hCG concentration which could be employed include HPLC, Western blotting, SDS-PAGE, enzyme or fluorescent immunoassay, but, as mentioned previously, preferred measurement techniques employ an immunochromatographic lateral flow type device, which is cheap yet sensitive and easy to use.

In another aspect, an apparatus is disclosed for determining the EDD and/or estimated date of conception. The apparatus is typically suitable and adapted for use in the method(s) defined previously. More specifically, the apparatus for use in determining the EDD and/or estimated date of implantation and/or date of conception for a human fetus present in a pregnant woman may include a data processor to process the hCG concentration data according to a suitable algorithm programmed into the apparatus so as to determine an estimated date of implantation and/or an estimated due date and/or an estimated date of conception. The hCG concentration data may be provided to the apparatus e.g. by inputting data from a user interface, such as a keyboard. Alternatively, the apparatus may include a system for measuring the concentration of hCG in a sample of body fluid obtained from the woman, so as to provide hCG concentration data, which can then be directly processed by the data processor, without the data needing to be input by a user. Exemplary systems are disclosed, for example, in U.S. Provisional Patent Application Ser. No. 60/508,001, filed Oct. 2, 2003, U.S. patent application Ser. No. 10/741,416 (U.S. 2005-0037510 A1), filed Dec. 19, 2003, U.S. patent application Ser. No. 10/742,459 (U.S. 2005-0037511 A1), filed Dec. 19, 2003, and U.S. patent application Ser. No. 10/816,216 (U.S. 2005-0036148 A1), filed Apr. 1, 2004. The aforementioned patent applications are hereby incorporated herein in their entireties by this reference.

The apparatus additionally may include a display to show the determined EDD and/or estimated date of implantation and/or estimated date of conception. The display may include an electronic alphanumeric display such as, for example, a liquid crystal display. The apparatus may include a data export component, for exporting the determined EDD and/or date of conception data to some other piece of equipment, such as a personal computer provided with a separate display.

In preferred embodiments, an apparatus may take the form of a kit including an electronic assay result reading device (similar to the CLEARBLUE EASY® Digital pregnancy test device, available from Unipath Limited, Bedford, UK) for use with disposable lateral flow assay test sticks. The test sticks themselves will be contacted with a urine sample from a woman and inserted into the electronic result reading device which, typically, will use an optical system to measure the amount of label accumulated in the test zone of the test stick (e.g. a transmission or reflectance reading to measure the amount of light respectively transmitted through, or reflected from, the test zone which in turn depends on the amount of label deposited in the test zone). The reading device can include a data processor which, from the measurements, will determine the concentration of hCG in the sample and process the hCG concentration data according to an algorithm programmed into the device, thereby to arrive at an EDD and/or an estimated date of conception (conveniently by determining, as an intermediate step, an estimated date of implantation).

Advantageously the data processor will include a microprocessor. If the device is to be used in a manner in which hCG tests are conducted on a plurality of days, it will be desirable for the reader to include some sort of calendar (preferably in electronic form) and a data storage device to store the results of previous hCG tests. The reader will preferably also include an integral power source, such as button-type lithium cell or other battery, or possibly a solar powered device such as a photovoltaic cell.

Test sticks for use with assay result reader may be generally conventional. However, as mentioned previously, the hCG testing regime is preferably able to provide accurate results over a wide range of different hCG concentrations. Conventional hCG-specific test sticks are not normally configured to provide for this possibility because, in a pregnancy test, a qualitative result is all that is required.

One solution to this problem would be to provide a number of different test sticks say, for example, a “low”, “medium” and “high” stick; the “low” stick being for use in tests conducted shortly after implantation and for use in an hCG concentration range of 10-100 mIU/ml; the “medium” stick being for use in an hCG concentration range of 100-1,000 mIU/m and the “high” stick, for use near the end of the testing window, at an hCG concentration range of 1,000-10,000+mIU/ml.

An alternative, and preferable, solution is to provide a single test stick which is modified so as to be able to provide an accurate result over a wide range of hCG concentration values. One way in which an otherwise conventional test stick might be modified to extend the concentration range over which it can provide an accurate quantitative result is to provide a plurality of test zones, e.g. a “low”, “medium” and “high” which correspond to the concentration ranges identified above.

By way of explanation, a single test stick could be provided with an immobilized capture reagent deposited in three discrete test zones: when the hCG concentration is low (early after implementation), substantially all of the hCG will be captured (directly or indirectly) at the “low” test zone furthest upstream. Later samples, containing a higher concentration of hCG, will tend to saturate the “low” test zone, so that significant amounts of hCG will be captured at the “medium” test zone. Later still in the testing window, when samples contain an even higher concentration of hCG, the “medium” test zone will also become saturated and significant amounts of hCG will be captured at the “high” test zone. The reading device will be programmed to distinguish between different zones, e.g. on a positional/spatial basis, so as to determine an accurate result for hCG concentration.

Although the above description refers to the use of three test zones, it should equally be possible to employ a test stick containing, say, two or four hCG concentration test zones, and indeed a two test-zone stick may be preferred as being simpler and cheaper to make and use.

In some embodiments, various aspects may also include measurement of luteinizing hormone (LH) in samples of body fluid (preferably urinary LH). Measurement of LH on a frequent basis can facilitate detection of the LH “surge” which occurs immediately prior to ovulation. Knowledge of the occurrence of ovulation can enable the user to estimate when implantation might take place (approximately 7 days later) and thus when to start testing for hCG. Again, methods of testing urinary LH concentration are well known to those skilled in the art, and convenient methods include the use of lateral flow immunochromatographic devices (e.g. as disclosed in EP 0291194). Thus, in one embodiment, a kit may include one or more test sticks for measurement of hCG in a sample of body fluid; one or more test sticks for measurement of LH in a sample of body fluid; and an assay result reading device equipped with a suitably programmed data processor and a display; the reading device being capable of detecting an LH surge in the LH test results obtained from the sample/s of body fluid and calculating therefrom an hCG test commencement day on which to start testing samples for hCG concentration and indicating the hCG test commencement day to the user via the display; and wherein the reading device is also programmed to determine and display an EDD and/or estimated date of conception derived from hCG concentration data.

The kit may be provided with more than one type of test stick, such as two types of test stick: one type for testing LH; and a second type for testing hCG. Alternatively the kit may be provided with a single type of test stick which can be used to determine both LH and hCG concentration (e.g. from a single urine sample applied to the stick).

The disclosed methods and apparatuses can be used for a number of purposes. For example, anxious expectant mothers can be reassured early in pregnancy that the pregnancy is proceeding normally, by measurement of hCG and calculation of the EDD. This applies especially to women experiencing their first pregnancy or who have experienced repeated miscarriage. In some instances, determining an estimated date of conception may help to identify the likely father. In addition, unusual hCG concentration data may be used to obtain an early indication of possible problems (e.g. ectopic pregnancy, miscarriage) or of multiple conceptions.

For the avoidance of doubt it is hereby explicitly stated that any feature described as “preferred”, “advantageous”, “desirable” or described in similar such terms may be employed in any embodiment in isolation or in combination with any other feature or features so described, insofar as the context permits.

The terms “comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

### EXAMPLES

### Example 1

As part of a larger study into the changes in concentration of various hormones during the female reproductive cycle, samples were gathered from a group of women volunteers. Early morning urine samples were collected on a daily basis, and these were analyzed, using standard immunological assay techniques, to obtain values for the concentration of a number of analytes, including urinary hCG, LH, follicle stimulating hormone (FSH) etc.

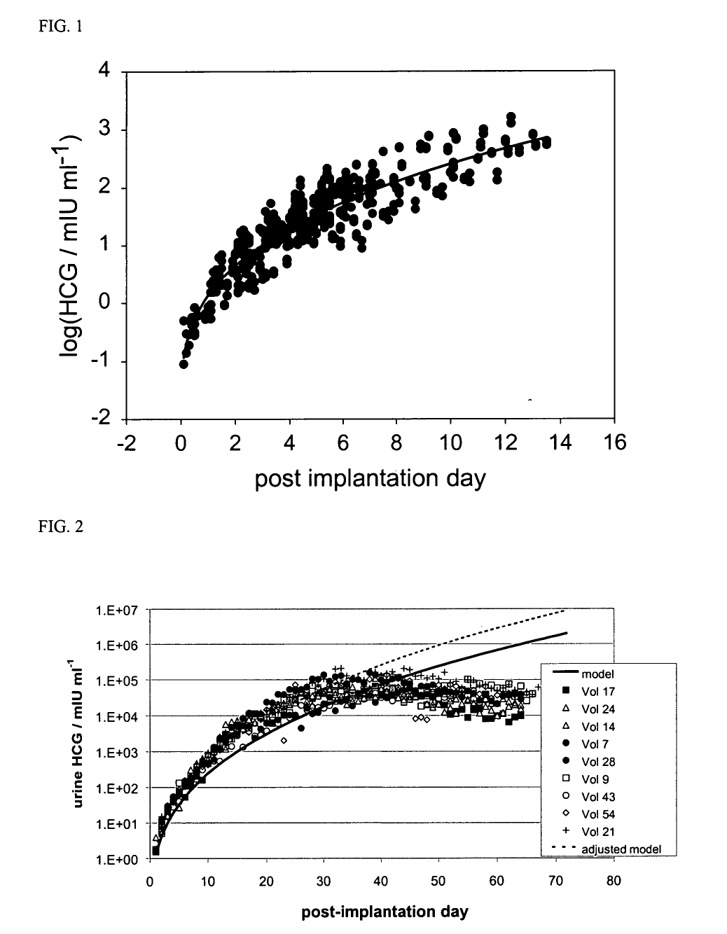

Subsequent analysis of the data showed that, in those women who became pregnant during the course of the study, the urinary concentration of hCG increased rapidly, as expected. Surprisingly, however, the inventor discovered that the graphs of hormone concentration vs time could be shifted on the time axis so as to almost superimpose. Namely, the progression of hCG increase between different individuals did not alter greatly and the difference between different individuals being to a significant extent due to the differences in the time at which the hCG concentration started to rise. The time at which this occurs is taken as the estimated time at which the embryo was implanted in the uterus. Typical results are shown in FIG. 1, which is a graph of the log of hCG concentration (in mIU/ml) against time (days post implantation). It can be seen that the individual data points cluster close to an empirically derived regression curve.

FIG. 2 shows similar data, plotted as log of urinary hCG concentration (in mIU/ml) against time (days post-implantation), but continued over a longer period at the start of the pregnancy. This figure shows that after about 25 days post-implantation, the rate of increase of hCG concentration substantially decreases from the logarithmic rate of increase exhibited earlier, and by about 35 days post-implantation the concentration of hCG reaches a plateau, and the disclosed methods can no longer be employed with confidence, because it will not be possible to determine accurately the date of implantation and hence the EDD or estimated date of conception.

### Example 2

From the data, the inventor was able to derive a number of algorithms which could be used to estimate the day of implantation based on measured hCG concentration values. Using the data illustrated in FIG. 1, the following equation was derived:
log h=a(t−t₀)⁰³³−1.86 (equation 1)
where h is the concentration of hCG in the sample expressed in mIU/ml, t-t₀ is the number of days between the date of measurement and the implantation date, and a is a coefficient (in this instance 1.99).

From the data illustrated in FIG. 2, which were obtained using a slightly different hCG assay technique, the value of a was found to be 2.15. The value of the parameter a has a value that is characteristic of the assay system being used and typically depends on the particular detection technique used (such as a particular antibody) and/or the particular form of hCG being detected. Thus, the selected system should be calibrated against known standards (such as samples obtained from pregnant females whose stage of gestation is precisely known based on the date of ovulation). For the assay systems described herein, the parameter a can have a value in the range of about 1.8 to about 2.3, more specifically about 1.91 to about 2.23, yet more specifically in the range of about 1.99 to about 2.15. The measurements reported herein were obtained by sandwich assay with antibodies recognizing the alpha and beta chains of hCG, using a DELFIA assay system from Perkin Elmer. The value 1.99 was obtained using Unipath antibodies, clones 3299 and 3478. The value 2.15 was obtained using Perkin Elmer antibodies.

From this, it follows that the equation to calculate the time (in days) which has elapsed since implantation, given a single measured value for hCG concentration is

$$t - {t}_{0} = \frac{{( log h + 1.86 )}^{3.0}}{a} \qquad ( equation 2 )$$

The estimated date of implantation can then be calculated by subtracting the number of days t-t₀ from the date on which the sample is taken or measured.

From the calculated day of implantation, the EDD is determined by adding about 37 weeks or about 259 days. The date of conception is estimated by subtracting 5 to 9 days, preferably 7 days (+/−2 days) from the date of implantation. The date of conception can be reported as 7 days before the calculated date of implantation with a margin of error of +/−2 days. Alternatively, the date of conception can be reported as a range from 5 to 9 days before the calculated date of implantation or as one of those dates.

### Example 3

A cause of uncertainty in the calculation of the date of implantation is that the value of the parameter ‘a’ varies from person to person (although by less than might have been predicted prior to the present disclosure). Another cause of uncertainty is that the urinary hCG concentration fluctuates randomly about the mean value throughout any given day. The inventor investigated whether these causes of uncertainty can be reduced by using a number of hCG concentration determinations (e.g. two measurements, taken 7 days apart).

Attempts to estimate parameters a and to by direct solution of equation (2) using two different values of log(hCG) obtained on two different days failed, because cubing the value (log(hCG)−1.86) amplified the errors. Small errors in the estimation of a, caused by small fluctuations in log(HCG), caused large errors in the estimate of to. The following method seemed to work satisfactorily, however.

From equation 1, the slope of the log(hCG)−time curve is

$$\frac{ⅆ [ log h ]}{ⅆ t} = 0.33 {a ( t - {t}_{0} )}^{- 0.67} \qquad ( equation 3 )$$

Solving equation 2 for a and substituting the expression for a in equation 3 gives:

$$t - {t}_{0} = \frac{log h + 1.86}{3 \frac{ⅆ [ log h ]}{ⅆ t}} \qquad ( equation 4 )$$

If two values are taken n days apart, t₁ is the time of the first measurement, 0.5 log(h₁h₂) denotes the mean value of the logs of the two measurements, and log(h₂/h₁) denotes the difference between the logs of the two measurements, then

$$\begin{matrix}{t}_{1} - {t}_{0} + n / 2 \approx \frac{0.5 log ( {h}_{1} {h}_{2} ) + 1.86}{\frac{3 log ( {h}_{2} / {h}_{1} )}{n}} & ( equation 5 ) \\ {t}_{0} \approx n / 2 + {t}_{1} - \frac{0.5 log ( {h}_{1} {h}_{2} ) + 1.86}{\frac{3 log ( {h}_{2} / {h}_{1} )}{n} } & ( equation 6 )\end{matrix}$$

or

If three or more measurements are taken then a standard regression method (such as least squares, particularly simplex or nonlinearl squares) may be applied to equation 2 to obtain to without prior assumption as to the value of a other than constraints based on the expected range of values. If the estimated value of a falls outside the expected range or otherwise gives an insensible value for to (such as a physically impossible time or a time at which the subject was known not to be pregnant), then the algorithm can prompt for further measurements to improve the accuracy of the estimate or can alert for the need to explore potential complications of the pregnancy.

### Example 4

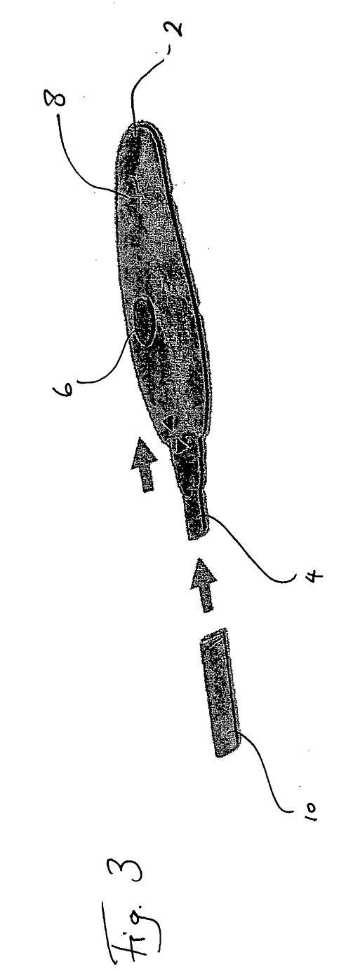

An apparatus as disclosed herein, and suitable for use in accordance with disclosed methods, takes the form of a kit including an electronic, battery powered assay result reading device and a plurality (typically three or four) test sticks for measuring the concentration of hCG in urine samples. The assay result reading device is closely similar to the CLEARBLUE EASY® Digital reading device commercially available from Unipath Limited (Bedford, UK) and is illustrated in FIG. 3.

The reading device includes an opaque housing 2 formed from a synthetic plastics material. At one end of the housing 2 is an aperture into which a test stick 4 can be inserted. Various guidance and positioning facilitators are provided, such that the test stick 4 can be placed into a precise location relative to the reader. The reading device further includes: an eject button 6, activation of which causes the test stick 4 (when read) to be ejected from the reading device; and an LCD window 8 which can be used to display information and messages. The test stick is provided with a removable cap 10.

Inside the opaque housing 2, and hence not visible in FIG. 3, are the optics for performing optical measurements (e.g. transmission or reflectance readings) on the test stick. Some exemplary optical systems are described in U.S. patent application Ser. No. 10/816,216 (US 2005-0036148 A1), filed Apr. 1, 2004. The optical measurements are typically dependent on the amount of label deposited in a test zone or zones on the test stick, which in turn depends on the concentration of analyte present in the sample applied to the test stick.

The reading device further includes a button-type flat lithium cell, a microprocessor, and a memory device. The microprocessor is programmed so as to calculate the hCG concentration from the optical measurements made, and additionally programmed with a suitable algorithm to use the derived hCG concentration data to determine the date of implantation, and hence the most likely date of conception and the estimated due date, which two pieces of information can be displayed in the liquid crystal display window 8.

The examples should not be construed as limiting in any way. The contents of all cited references are hereby expressly incorporated by reference.

1. A method of determining the estimated date of implantation of an embryo, comprising:
testing a sample of a body fluid from a pregnant human female subject so as to obtain data indicative of the concentration of human chorionic gonadotrophin (hCG) in the sample; and
calculating an estimated date of implantation from the hCG concentration data.

2. A method of determining the estimated due date for a pregnant human female subject, comprising:
determining the estimated date of implantation of an embryo as set forth in claim 1;
and calculating the estimated due date by adding about 37 weeks to the estimated date of implantation.

3. A method of determining the estimated date of conception of a human fetus, comprising: determining the estimated date of implantation of an embryo as set forth in claim 1; and calculating the estimated date of conception by subtracting 7 days +/−2 days from the estimated date of implantation.

4. A method according to claim 1, wherein the determination is made on the basis of a single hCG concentration measurement.

5. A method according to claim 1, wherein the determination is made on the basis of a plurality of hCG concentration measurements.

6. A method according to claim 1, wherein the hCG concentration data is obtained from a sample or samples collected in the time interval up to 15 days post-implantation.

7. A method according to claim 1, wherein the hCG concentration data is obtained from a sample or samples collected in the time interval up to 8 weeks following the female subject's last menstrual period.

8. A method according to claim 7, wherein the sample or samples is collected in the time interval up to 7 weeks following the female subject's last menstrual period.

9. A method according to claim 8, wherein the sample or samples is collected in the time interval up to 6 weeks following the female subject's last menstrual period.

10. A method according to claim 1, wherein the body fluid sampled is urine.

11. A method according to claim 1, wherein hCG concentration data is obtained by conducting a lateral flow immunoassay using the sample of body fluid.

12. A method according to claim 1, further comprising the step of monitoring the subject to detect ovulation or an event associated with ovulation.

13. A method according to claim 12, wherein the monitoring comprises measuring luteinizing hormone (LH) in a sample of body fluid or urine periodically collected from the subject.

14. The method of claim 1, wherein calculating the estimated date of implantation comprises calculating the time t-t₀ (in days) which has elapsed since implantation as being
$$t - {t}_{0} = \frac{{( log h + 1.86 )}^{3}}{a} ,$$
wherein h is the sample concentration of hCG in mIU/ml, and a has a value in a range characteristic of an assay system used to obtain the hCG concentration data.

15. The method of claim 14, wherein the assay system comprises an antibody recognizing at least one of the alpha and beta chains of hCG.

16. The method of claim 14, wherein calculating further comprises subtracting t-t₀ from the date on which the sample was taken.

17. The method of claim 14, wherein the processor calculates an estimated due date by subtracting t-t₀ from the date on which the sample was taken and adding about 37 weeks.

18. The method of claim 14, wherein the processor calculates an estimated date of conception by subtracting t-t₀ from the date on which the sample was taken and further subtracting 7 days +1-2 days.

19. The method of claim 1, wherein the hCG concentration data is obtained using an assay system comprising an antibody recognizing at least one of the alpha and beta chains of hCG.

20. The method of claim 1, wherein two samples are taken n days apart, and the estimated date of implantation to is calculated as being
$${t}_{0} \approx \frac{n}{2} + {t}_{1} - \frac{0.5 log ( {h}_{1} {h}_{2} ) + 1.86}{\frac{3 log ( {h}_{2} / {h}_{1} )}{n}}$$
wherein h₁ and h₂ are, respectively, the sample concentrations of hCG in mIU/ml for the first and second measurements, and t₁ is the time in days of the first measurement.

21. The method of claim 1, wherein three or more samples are taken, and the estimated date of implantation t₀ is calculated by applying a standard least-squares regression method to
$$t = \frac{{( log h + 1.86 )}^{3}}{a} + {t}_{0} ,$$
where, respectively for each sample, t is the time of the measurement in days, h is the sample concentration of hCG in mIU/ml, and a has a value in a range characteristic of an assay system used to obtain the hCG concentration data.

22. A method of detecting an abnormal pregnancy comprising:
obtaining at least two samples of a body fluid from a pregnant human female subject;
measuring the concentration of hCG in each sample using an assay system;
estimating the values of a and/or to by regression of the equation
$$t = \frac{{( log h + 1.86 )}^{3}}{a} + {t}_{0} ,$$
wherein, respectively for each sample, t is the tine of the measurement, and A is the sample concentration of hCG in mIU/ml; and
if a falls outside a range characteristic of the assay system or if t₀ has an insensible value, concluding that the pregnancy is abnormal.

23. A device for determining the estimated implantation date of an embryo, comprising: a data processor responsive to hCG concentration data to calculate the estimated implantation date.

24. A device according to claim 23, further comprising a measurement system for measuring the concentration of hCG in a sample of body fluid obtained from a subject, so as to provide hCG concentration data.

25. A device according to claim 23, further comprising a display to display the estimated implantation date of the embryo.

26. A device according to claim 23, further comprising a system for measuring the concentration of LH in a sample of body fluid obtained from a subject and, optionally, a display for indicating to a user when to conduct an assay to measure hCG concentration.

27. A device according to claim 23, further comprising one or more disposable, lateral flow assay test sticks and an electronic assay result reader.

28. Previously presented) A device of claim 23, wherein the data processor calculates the estimated date of implantation by calculating the time t-t₀ (in days) which has elapsed since implantation as being
$$t - {t}_{0} = \frac{{( log h + 1.86 )}^{3}}{a} ,$$
wherein h is the sample concentration of hCG in mIU/ml, and a has a value in a range characteristic of an assay system used to obtain the hCG concentration data.

29. A device according to claim 23, further comprising an optical detection system for measuring the signal.

30. A device according to claim 23, which is a lateral flow assay device.

31. A device for estimating the date of implantation of an embryo comprising: measuring means for obtaining data indicative of the concentration of hCG in a sample of a body fluid from a pregnant human female subject; and means for calculating the estimated implantation date from the hCG concentration.
